(19) United States
(12) Patent Application Publication
(10) Pub. No.: US 2021/0252663 A1
(43) Pub. Date: Aug. 19, 2021

(54) ABRASIVE PROCESSING OF INNER SURFACE OF SEAMLESSLY DRAWN TUBES INCLUDING MEDICAL DEVICE TUBES

(71) Applicant: BIOTRONIK AG, Buelach (CH)

(72) Inventors: Ullrich Bayer, Bad Doberan (DE); Sarah Dramm, Rostock Diedrichshagen (DE); Alexander Joern, Bad Doberan (DE); Bjoern Rueter, Osteebad Nienhagen (DE); Torsten Homuth, Osteebad Nienhagen (DE); Nils Froehlich, Rostock (DE)

(21) Appl. No.: 17/270,595

(22) PCT Filed: Aug. 29, 2019
(86) PCT No.: PCT/EP2019/073049

§ 371 Date: Feb. 23, 2021

(30) Foreign Application Priority Data
Aug. 30, 2018 (EP) 18191662.8

(51) Int. Cl. B24B 5/40; B24B 27/033; A61F 2/91; B24B 47/14

(52) U.S. Cl. CPC B24B 5/40; B24B 27/033; A61F 2/91; A61F 2230/0071; A61F 2250/0092; A61F 2230/0069; B24B 47/14

(57) ABSTRACT

A tool for processing an inner surface of a seamlessly drawn tube is sized and configured to be inserted into the interior of the tube. The tool has an outer side defining multiple flow channels in the form of grooves arranged adjacently to each other in a circumferential direction of the tool, wherein each groove on the outer side forms a cutting edge and preferably at least two cutting edges. The tool is moved along a longitudinal axis (x) of the tube while simultaneously rotating the tool in the circumferential direction (U) of the tool by applying a gaseous medium (G) to the tool and/or by acting on the tool with an alternating magnetic field to remove contaminations of the tube which protrude from the inner surface.

### PRIORITY CLAIM

This application is a 35 U.S.C. 371 U.S. National Phase and claims priority under 35 U.S.C. § 119, 35 U.S.C. 365(b) and all applicable statutes and treaties from prior PCT Application PCT/EP2020/073049, which was filed Aug. 29, 2019, which application claimed priority from European Application EP 18191662.8, which was filed Aug. 30, 2018.

### FIELD OF THE INVENTION

The present invention relates to a method and device for the abrasive processing of an inner surface of a tube, in particular a tube semi-finished product for a medical device, such as a tube used to produce a stent.

### BACKGROUND

Tubes or tube semi-finished products, which are used as starting material for medical devices, regularly fail to meet the demands in respect of the required geometries and the freedom from surface contaminations. Surface contaminations of this kind can be formed for example during production processes by residues of lubricants and other processing aids, and by deposits of tool surfaces on the tube inner surfaces.

Vascular vessel implants are generally produced by the laser cutting of seamlessly drawn tubes. The need for seamlessly drawn tubes results from the fact that the welding together of curved sheet material leads to indifferent semi-finished product properties—particularly in the weld seam—which prohibits their use for stents. This is due in particular to the demands with regard to the fatigue strength of implants that are stressed pulsatively.

Various technologies are used in the manufacture of seamlessly drawn tubes. Regardless of this, it is unavoidable that forming tools are used in order to set the desired tube dimensions, these tools having direct material contact with the tube and therefore being subjected to certain signs of wear on account of the forces occurring during the forming.

This wear can assume a material-removing (abrasive) or material-depositing (adhesive) character or combinations of the two. It is routine practice that such signs of wear can be minimised by the use of forming aids, such as lubricants. This, however, in turn poses the risk that lubricants will enter into a chemical interaction with the tube surfaces. These effects are increased by thermal processes (heat forming), which are utilised in most tube materials (on account of material-induced limited cold formability). Reaction products made of the tool materials (for example steel or hard metal) and a wide range of different lubricants, usually having a high carbon content, connect to the tube surfaces, resulting in the formation of deposits/bonds of different hardness adhering to the tube surfaces. These can assume different geometrical forms (individual particles, elongate islands, plateau areas). In most cases they are oriented in the tube longitudinal axis.

In order to attain a sufficient surface quality of the inner surface of the tube in question for the further processing to form a medical device, it is known in the prior art to process tube semi-finished products of the kind described at the outset by honing, lapping, pressure jet lapping, sandblasting, chemical removal by pickling solutions, rinsing with hydrocarbon-containing solutions with and without ultrasonic assistance or by interior brushes.

Here, however, honing has proven to be extremely difficult and complex in the case of tube lengths greater than 1 m. Furthermore, in the case of lapping and pressure jet lapping there is the risk that residues of the lapping agent will remain in the tube and then have to be removed again using liquid cleaning agents. Furthermore, in the case of lapping and pressure jet lapping the removal is performed preferably in the tube longitudinal direction, i.e. existing grooves running in the tube longitudinal direction are deepened rather than smoothed.

Furthermore, in the case of sandblasting the removal is likewise performed preferably in the tube longitudinal direction, i.e. here as well existing grooves are deepened rather than smoothed. Residues of the blasting material also often remain on the tube inner surface, become stuck there and remain partially interlocked and frictionally engaged in the rough tube inner surfaces, wherein complete removal of the blasting material by subsequent processes (for example by pickling or electropolishing in the case of stent production) is not guaranteed. It is also not to be ruled out that contaminations might even be pressed further into the tube surface by the sandblasting process.

With regard to the chemical removal by pickling solutions, a non-uniform and decreasing pickling effect is frequently to be expected over the tube length, which can lead to undesirable differences in wall thickness between the tube start, tube middle and tube end. Due to the high chemical resistance of conventional tube materials, highly effective and extremely aggressive pickling solutions have to be used, which additionally are pushed through the tube at high pressure. This leads to a high outlay in respect of industrial safety and environmentally friendly disposal of the spent pickling solutions.

Lastly, the interior brushes have the primary disadvantage that the pull-through forces created by the internal friction of the bristles against the tube inner wall are very high in the case of long tubes. This friction can be minimised only by reducing the difference between the brush outer diameter and the tube inner diameter or by tailored brush geometries. Both solutions lead to an insufficient cleaning effect of the tube inner surfaces.

Furthermore, a processing by magnetic grinding is described in Junmo Kang et al., Procedia CIRP 1 (2012) 414-418, in which the simultaneous processing of a number of regions in capillary tubes is possible by a multi-pole system with use of a partially heat-treated magnet tool.

Proceeding from the above-presented prior art, the object of the present invention is to create a method and a device which make it possible to remove the foreign materials not corresponding to the required tube composition in a residue-free manner. In particular, special attention should be paid to ensure that the original tube geometry is not negatively influenced, as is the case for example when cleaning media are deployed only in the direction of the tube longitudinal axis.

### SUMMARY OF THE INVENTION

A tool for processing an inner surface of a seamlessly drawn tube is sized and configured to be inserted into the interior of the tube. The tool has an outer side defining multiple flow channels in the form of grooves arranged adjacently to each other in a circumferential direction of the tool, wherein each groove on the outer side forms a cutting edge and preferably at least two cutting edges. The tool is moved along a longitudinal axis (x) of the tube while simultaneously rotating the tool in the circumferential direction (U) of the tool by applying a gaseous medium (G) to the tool and/or by acting on the tool with an alternating magnetic field to remove contaminations of the tube which protrude from the inner surface.

### BRIEF DESCRIPTION OF THE DRAWINGS

Embodiments and also features and advantages of the invention will be explained hereinafter with reference to the drawings, in which:

FIG. 1 shows a perspective view of an embodiment of a tool according to the invention which is spherical;

FIG. 2 shows a perspective view of a further embodiment of a tool according to the invention which is cylindrical;

FIG. 3 shows a possible detail of the tool shown in FIG. 2;

FIG. 4 shows a schematic view of an embodiment of a device according to the invention with a tool which is formed in particular in the manner of the embodiment shown in FIG. 1;

FIG. 5 shows a perspective view of the tool shown in FIG. 2, which is arranged in a tube in order to process, in particular to clean, the inner surface of the tube; and

FIG. 6 shows a further embodiment of a device according to the invention with a tool in the manner of FIG. 2.

### DETAILED DESCRIPTION OF THE PREFERRED EMBODIMENTS

A tool for processing an inner surface of a seamlessly drawn tube is sized and configured to be inserted into the interior of the tube. The tool has an outer side defining multiple flow channels in the form of grooves arranged adjacently to each other in a circumferential direction of the tool, wherein each groove on the outer side forms a cutting edge and preferably at least two cutting edges. The tool is moved along a longitudinal axis (x) of the tube while simultaneously rotating the tool in the circumferential direction (U) of the tool by applying a gaseous medium (G) to the tool and/or by acting on the tool with an alternating magnetic field to remove contaminations of the tube which protrude from the inner surface.

The tool is characterised in particular in that, in the interior of the tube to be processed, the tool is not moved by a rigid coupling, for example in the form of a shaft, which mechanically connects the tool to an actuator arranged outside the tool. Rather, the tool is merely arranged or can be arranged completely in the interior of the tube and is moved there by application of pressure or negative pressure and/or by an alternating magnetic field.

The separated contaminations or particles can be expelled from the interior of the tube in particular via the flow channels, for example by the gaseous medium. Should an alternating magnetic field be used in order to move/rotate the tool, for example a coil movable along the tube can be used, with a current flowing in said coil for generation of the alternating magnetic field. The coil can surround the tube.

The operating principle of the present invention is therefore based in particular on an inner tool with cutting edges and in particular a hard, wear-resistant and rough outer side. The outer diameter of the tool is preferably tailored to the particular inner diameter of the tube to be processed.

In order to attain the desired inner processing effects, the tool is in particular able to move in the interior of the tube both radially and in the direction of the longitudinal axis of the tube. This multi-axial movement can be achieved by different operating principles. It is achieved on the one hand by a preferably alternating negative pressure or positive pressure and a fluidically optimised surface structuring of the outer side of the tool by the flow channels. Alternatively, the tool is also set in motion by a magnetic field. This can be achieved for example with the aid of a coil guided externally around the tube. The coil can be displaced over the entire tube length, and the tool rotating in the interior in a kind of tumbling movement can follow this movement. The required removal of the inner surface of the tube can be varied by local residence times, the rotational speed of the tool in the circumferential direction of the tool, and the abrasive effect of the outer side of the tool or of the cutting edge(s).

The invention thus makes it possible to provide tube semi-finished products in particular for stent production (coronary, peripheral or TAVI stents), which products lead to a significantly reduced defect rate in respect of the inner surface condition. In particular, foreign materials on the inner surface of the tube can be removed without leaving behind any residue, wherein draw marks are avoided or draw marks already resulting from the tube production process are levelled. The invention thus allows an improvement of the long-term fatigue properties of stents produced from treated tubes. Furthermore, the post-processing outlay for laser-cut stents can be reduced. Lastly, on account of the invention, there is also the possibility to introduce a locally modified/delimited tube inner diameter.

The flow channels on the outer side of the tool, during the processing method according to the invention, ensure a movement of the tool during which movement components along the longitudinal axis of the tube to be cleaned are superimposed by movement components along the circumferential direction of the tube to be cleaned, in particular by the rotation of the tool about itself. The deposits in the interior of the tube to be processed are thus removed not only along the tube axis. The movement pattern formed from the superimposing movement components automatically also results in a removal in the radial direction. Grooves running in the tube longitudinal direction are thus avoided and deposits are removed much more reliably. The described superimposition of the axial and radial movement components is provided here regardless of whether the tool is moved by an application of pressure or negative pressure and/or by an alternating magnetic field. The flow channels on the outer side of the tool are responsible for the movement pattern. They are designed as described at the outset. A design of this kind ensures the superimposition of the axial and radial movement and in particular the inherent rotation of the tool.

In the method according to a preferred embodiment the tool is moved along the longitudinal axis and in so doing rotates in the circumferential direction or about a rotation axis such that it performs a tumbling movement.

With regard to the movement of the tool along the longitudinal axis of the tube and the rotation of the tool in the circumferential direction, it is preferably provided in accordance with an embodiment of the method that the flow channels each have a curved profile. As a result, as the gaseous medium flows through the flow channels, a rotation of the tool in the circumferential direction of the tool and an advancement of the tool in the direction of the longitudinal axis are produced.

In accordance with one embodiment of the invention it is provided that the tool is spherical or ellipsoidal, in particular is formed as a spheroid. Here, the tool can have in particular a cylindrical symmetry with respect to the above-mentioned axis of rotation about which the tool rotates.

It is furthermore provided in accordance with an alternative embodiment of the method that the tool is cylindrical, wherein in particular said rotation axis about which the tool is rotated is a cylinder axis of the tool. On account of the tumbling movement of the tool when this is moved back and forth and at the same time rotated, the position of the rotation or cylinder axis of the tool varies by the position of the longitudinal axis of the tube.

In particular in the case of a cylindrical tool it is provided that the flow channels each extend in a direction which runs skewed relative to the cylinder axis of the tool.

It is furthermore provided in accordance with an embodiment of the method that the flow channels or grooves have a circle segment shape in cross-section. As a result, two sharp cutting edges are provided at the transition or opening of each flow channel to the outer side of the tool. The grooves, however, can also have different cross-section shapes.

It is furthermore provided in accordance with an embodiment of the method that the outer side of the tool has a surface structure which is designed to act abrasively on the inner surface of the tube, wherein, as the tool moves and rotates in the interior of the tube, contaminations of the tube which protrude from the inner surface of the tube are removed with the aid of the surface structure.

It is furthermore provided in accordance with an embodiment of the method that the tool, during the movement along the longitudinal axis, is moved back and forth as a result of sides of the tool facing away from one another in the interior of the tube being acted on by different pressures on or being acted on differently by a gaseous medium, or as a result of the alternating magnetic field or said coil being moved back and forth accordingly along the longitudinal axis with respect to the tube. With the use of a gaseous medium (for example pressurised air) it is possible for example for a negative pressure to be applied to the interior of the tube at one opening of the tube in alternation with a positive pressure applied to the interior of the tube at the opposite opening. The tool disposed in-between then moves accordingly toward the negative pressure, such that it is movable back-and-forth in the interior.

It is furthermore provided in accordance with an embodiment of the method that the tool comprises at least one permanent magnet for the movement and rotation of the tool by the alternating magnetic field. For example, two permanent magnets can be used, which can be incorporated in the tool and for example can extend one on each of the two sides of the rotation axis, parallel to the rotation axis, such that the rotation axis runs between the two permanent magnets.

It is furthermore provided in accordance with an embodiment of the method that a wall thickness of the tube varying along the longitudinal axis is produced by the tool in that the tool (for example by the coil or corresponding application of pressure) is positioned in a defined manner along the longitudinal axis in the interior of the tube and is rotated in the particular position.

It is furthermore provided in accordance with an embodiment of the method that the tube is made from a metal alloy, in particular a nickel-titanium alloy (for example nitinol) or a cobalt-chromium alloy or a chromium-nickel steel.

It is furthermore provided in accordance with an embodiment of the method that the tube has an inner diameter in the range of from 1.5 mm to 10 mm.

It is furthermore provided in accordance with an embodiment of the method that the tube has a length of at least 1 m, in particular of at least 1.5 m, in particular of at least 2 m. The length of the tube can lie for example in the range of from 1 m to 5 m, in particular in the range of from 1 m to 3 m, in particular in the range of from 1.5 m to 2.5 m.

It is furthermore provided in accordance with an embodiment of the method that the tube is a blank for a medical device, in particular a blank for a medical implant, in particular a blank for a stent or a support (for example frame) or a cardiac valve (for example TAVI) or a cannula for injection needles that is subject to the highest requirements in respect of cleanliness.

In accordance with a further embodiment of the invention it can be provided that the blank processed by the tool is further processed to form a medical implant (for example stent or scaffold) or to form a component (for example stent or scaffold) of a medical implant.

A further aspect of the present invention relates to a device for processing an inner surface of a tube extended along a longitudinal axis, wherein the inner surface faces an interior of the tube or delimits said interior, and wherein the device at least comprises: a tool, which is designed to be inserted into the interior of the tube, wherein the tool has an outer side in which there are formed multiple flow channels in the form of grooves, which are arranged adjacently in a circumferential direction of the body, and wherein each groove on the outer side forms at least two cutting edges, which in particular are designed to remove contaminations which protrude from the inner surface of the tube.

In accordance with an embodiment of the device it is provided that the flow channels or grooves extend over an entire length of the tool in the axial direction (i.e. along the rotation axis of the tool).

It is furthermore provided in accordance with an embodiment of the device that the device has a movement-generating apparatus which is configured to move the tool in the interior of the tube along the longitudinal axis of the tube, in particular to move it back and forth, and at the same time to rotate it in the circumferential direction of the tool.

In this regard it is provided in accordance with an embodiment of the device that the movement-generating apparatus is designed to act on the tool with a pressure or a gaseous medium such that the tool is moved in the interior of the tube along the longitudinal axis, in particular is moved back and forth, and at the same time is rotated in the circumferential direction.

It is furthermore provided in accordance with an embodiment of the device that the device has a movable coil, which is designed to generate an alternating magnetic field at the location of the tool (when the tool is arranged in the interior of the tube to be processed) in order to move the tool along the longitudinal direction of the tube (in particular back and forth) and/or to rotate it in the circumferential direction. In this embodiment the tool preferably also comprises at least one permanent magnet.

In this embodiment the arrangement of the permanent magnet(s) in the tool, the externally applied frequency of the alternating magnetic field, and the feed of the movable coil in the longitudinal direction of the tube determine the inherent rotational speed (rotary speed) of the tool in the tube and the speed of the tool in the longitudinal direction. In this embodiment of the invention the individual contributions of the superimposed movement components along the longitudinal axis of the tube to be cleaned and along the circumferential direction of the tube to be cleaned thus can be controlled very well and precisely.

It is furthermore provided in accordance with an embodiment of the device that the flow channels each have a curved profile along the outer side of the tool (see also above).

It is furthermore provided in accordance with an embodiment of the device that the tool is spherical.

It is furthermore provided in accordance with an embodiment of the device that the tool body is cylindrical.

It is furthermore provided in accordance with an embodiment of the device that the flow channels each extend in a direction that is skewed relative to a cylinder axis of the tool.

It is furthermore provided in accordance with an embodiment of the device that the flow channels have a circle segment shape in cross-section, such that said cutting edges are provided in particular on the outer side of the tool.

It is furthermore provided in accordance with an embodiment of the device that the outer to side of the body has a surface structure which is designed to act abrasively on the inner surface of the tube.

It is furthermore provided in accordance with an embodiment of the device that the body comprises at least one permanent magnet for the movement and rotation of the body by the alternating magnetic field (see also above).

In accordance with a further aspect of the invention a medical implant is disclosed, comprising at least one portion of a tube which has been processed by the method according to the invention.

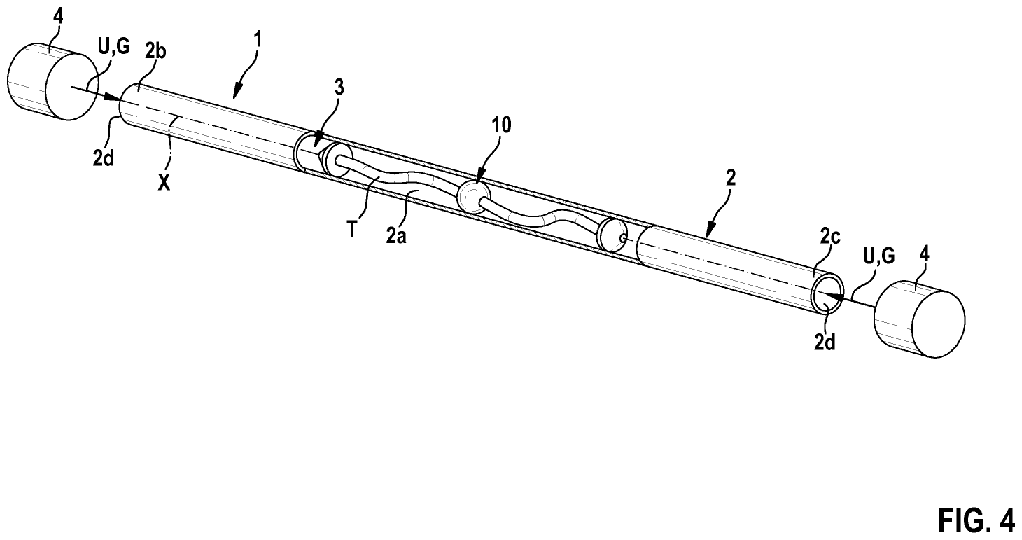

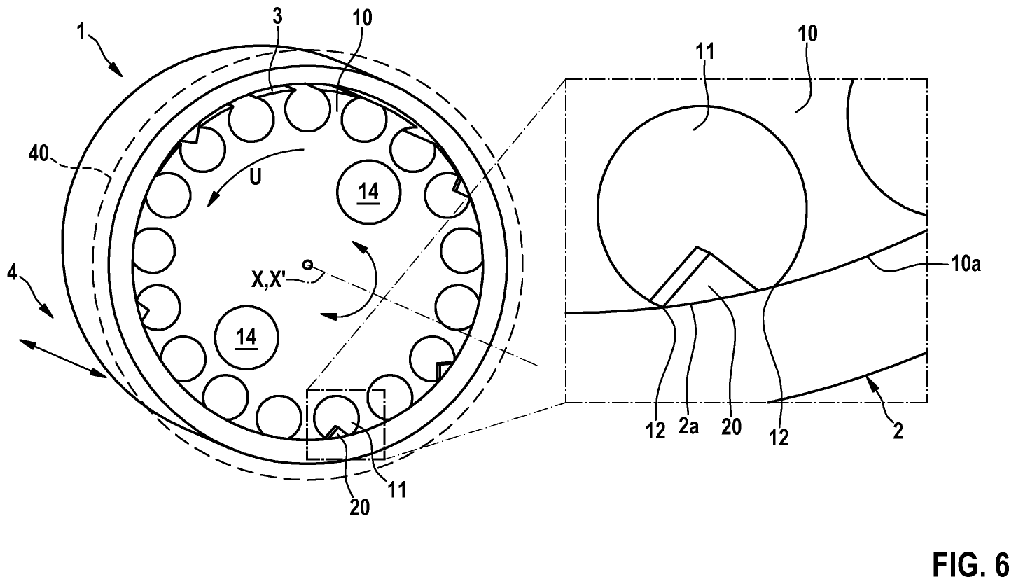

A preferred embodiment of the present invention relates to a method for processing an inner surface 2a of a tube 2 extended along a longitudinal axis x, wherein the inner surface 2a faces an interior 3 of the tube 2, and wherein the method has at least the following steps: inserting a tool 10 into the interior 3 of the tube 2, wherein the tool 10 has multiple flow channels 11, which are open to the outer side 10a, in the form of grooves 11, which are arranged adjacently in a circumferential direction U of the tool 10, and wherein each groove 11 forms preferably two cutting edges 12 on the outer side 10a, and moving the tool 10 in the interior 3 of the tube 2 along the longitudinal axis x of the tube 2 and simultaneously rotating the tool 10 in the circumferential direction U of the tool 10, for example by applying a pressure or a gaseous medium to the body (see for example FIG. 4) and/or by acting on the body with an alternating magnetic field (see for example FIG. 6), wherein contaminations 20 of the tube 2 which protrude from the inner surface of the tube or material regions 20 of the tube 2 are separated by the cutting edges 12. The contaminations are in particular undesirable adhesions/foreign materials of the tube 2. However, they can also be unevennesses/protrusions of the inner surface 2a of the tube 2 or the actual tube material.

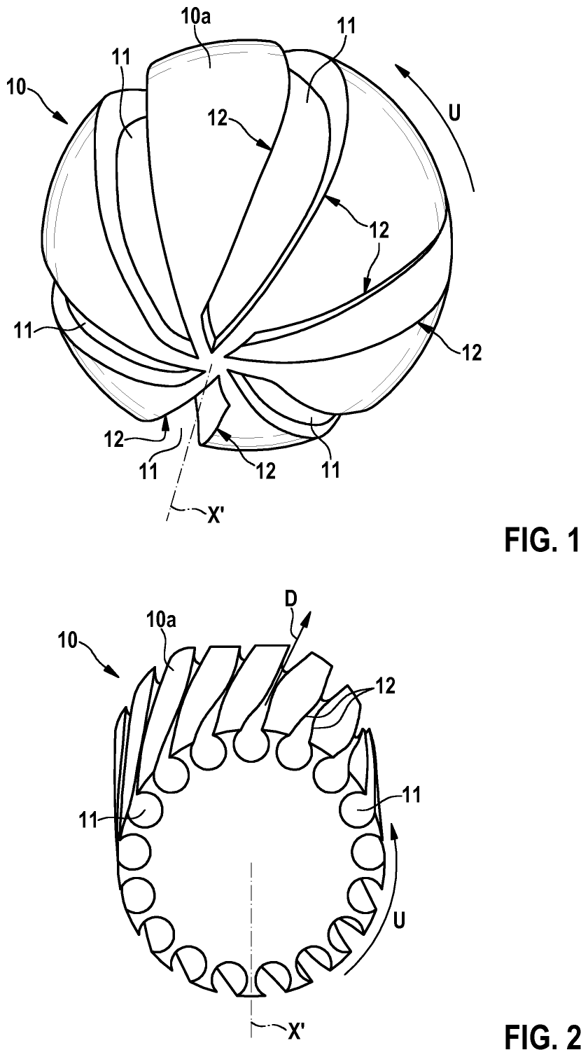

Additional preferred features of the tool are shown in FIGS. 1 to 3, 5 and 6.

The solution according to the invention is aimed at in particular at least one tool 10, which is also referred to as an inner tool 10, since it is arranged completely in the interior 3 of the tube 2 in order to abrasively remove adhering foreign materials 20 of the tube 2 situated in the tube interior 3. The abrasive effect can be achieved both with the principle of the geometrically specified edge, which is formed here by the cutting edges 12, and with the principle of the geometrically unspecified edge. For example, this can be a surface structure 13 of the outer side 10a of the tool 10.

The inner tool 10 preferably has a defined smaller outer diameter as compared to the tube inner diameter. The outer shape of the inner tools can be both spherical according to FIG. 1 and cylindrical according to FIG. 2. The tools 10 can furthermore be manufactured for example from a pickled hard metal.

The inner tools 10 additionally preferably have a defined surface structure so as to be able to efficiently remove contaminations 20 from the tube inner surface 2a.

In accordance with an embodiment a macroscopic structure of the outer tool form in the form of sharp-edged, hard and cutting-edge-containing flow channel edges 12 is provided here in accordance with FIGS. 1 and 2. The flow channels or grooves 11, which for example in accordance with FIGS. 1 and 2 run in a curved manner over the sphere circumference or the cylinder circumference, are also in each case preferably bifunctional elements, which, when different air pressures are applied at the tube ends or openings 2b, 2c of the tube 2, generate both a rotation in the circumferential direction U of the tool 10 in question (or rather about a rotation axis x′ of the tool 10 in question) and a movement along the longitudinal axis or tube axis x.

The cutting edge or flow channel edge 12, upon contact with the inner surface 2a of the tube 2 affected by foreign material, brings about a material-removing effect, without the rest of the tube inner wall being subjected to a significant material removal.

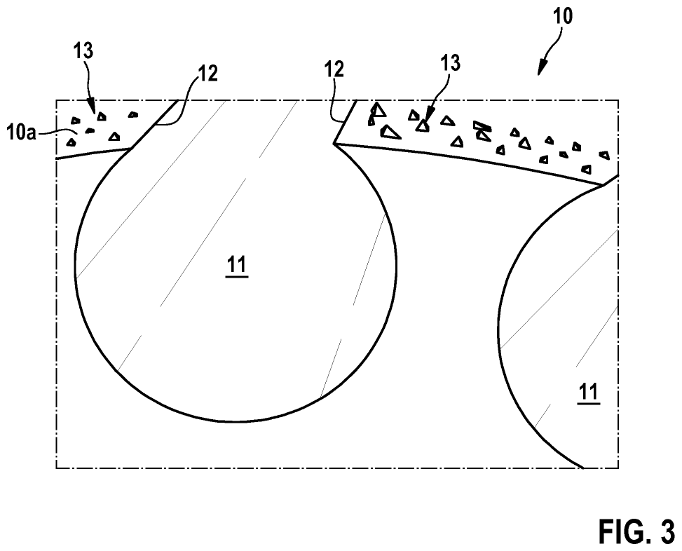

The tools 10, as shown with reference to FIG. 3, can also have a microscopically roughened surface structure 13 of the outer side 10 (for example a microstructure), which enables a further smoothing of the tube inner surfaces or inner surface 2a. An additional abrasive effect is attained by the microscopically roughened surface. Surfaces of this kind preferably have a mean rough value in the range of from 0.006 to 0.2 (specified using a stylus instrument). The risk of a local roughening is thus minimised, and the requirement of a smooth tube inner surface free from micro-cracks and micro-notches is satisfied. As shown in FIG. 3 the surface structure 13 can be generated by a pickling process, which roughens the hard metal surface 10a. The surface structure 13 can then be formed for example by exposed tungsten carbide particles. The roughness of the tool surface is influenced by the degree of exposure of the tungsten carbide particles and the mean size of the tungsten carbide particles. The roughness of the tube surface can thus be varied.

The flow channels 11, as can be seen for example with reference to FIGS. 2 and 3, preferably also have an opening at the outer diameter or at the outer side 10a, which opening tapers on both sides to a cutting edge 12 (for example following the form of a milling head). The flow channels 11 can have a circle segment shape in cross-section. The edge 12 is in each case designed to separate foreign materials 20 protruding from the tube inner surface or from the inner surface 2a of the tube (for example elevations, plateau areas, etc.). The resultant particles are suctioned away in the relevant flow channel 11 for example by way of the pressure differences (for example by the application of a gaseous medium, in particular pressurised air, to the tool 10).

FIG. 4 shows schematically a trajectory T of the tumbling movement of the spherical tool 10 according to FIG. 1 in the interior 3 of the tube 2 (for example with suctioning by negative pressure from the right-hand tube side 2c).

In accordance with an embodiment of the invention, for example a tube 2 made of the alloy nitinol according to ASTM F2063 with an outer diameter of 7.000 mm and a wall thickness of 0.500 mm is processed by the method according to the invention. The tube inner diameter is thus 6.000 mm. The tube inner surface or inner surface 2a of the tube 2 has undesirable plateau-like elevations 20, which for example consist of residues of baked-on tube drawing aids. These contaminations 20, which have a high carbon content, have a high adhesion to the tube inner surface 2a and cannot be removed by conventional cleaning methods without leaving behind any residue. In a tube 2 of this kind, approximately 2 m long, a spherical tool 10 according to FIG. 1 is inserted. The outer diameter of this sphere or this tool 10 is preferably 5.950 mm. Pressure apparatuses 4 are attached at both tube ends 2b, 2c of the tube 2 (see FIG. 4) and form a movement-generating apparatus and are designed to always apply a gaseous medium Gin the form of pressurised air at one tube end 2b, 2c and to apply a vacuum or negative pressure U at the other end 2c, 2b in alternation. Flexible and air-permeable reticular lattices (filters) 2d, which for example can be made of a plastic (mesh width approximately 0.5 mm), are preferably situated at both tube ends 2b, 2c. These prevent an accidental escape of the spherical tool 10 from the tube interior 3.

The application of different pressures by the movement-generating apparatus or pressure apparatuses 4 causes the sphere 10 to move back and forth. In so doing, the flow channels cause a simultaneous rotation of the sphere 10. In the event that any unevennesses/contaminations 20 are encountered, the sharp-edged flow channel or cutting edges 12 shear off the unevennesses or contaminations 20, and the abraded particles are suctioned away to the tube ends 2b, 2c via the flow channels 11. The mesh width of the filter 2d is in particular selected such that these particles can be suctioned away from the tube interior 3 without resistance, whilst the sphere or the tool 10 remains within the tube interior. The frequency or the speed with which the sphere or the tool 10 moves in the interior 3 of the tube 2 can be adjusted via the frequency of the pressure/vacuum application G, U. With a speed of approximately 1 m/s to 5 m/s in the direction of the longitudinal axis x of the tube 2, the spherical tool 10 rotates with a number of revolutions per second about its rotation axis x′. After a residence period of the sphere 10 in the tube 2 of approximately 1 min it is ensured that each point in the tube 2 has come into contact with the sphere 10 at least 3 times. The tube inner surface or inner surface 2a of the tube 2 is then in a cleaned state and is free from contamination by foreign material. After the subsequent processes (laser cutting, pickling, electropolishing), vascular implants produced in this way are characterised by defect-free inner surfaces. This leads to an increased long-term fatigue resistance and ultimately to an increased product reliability.

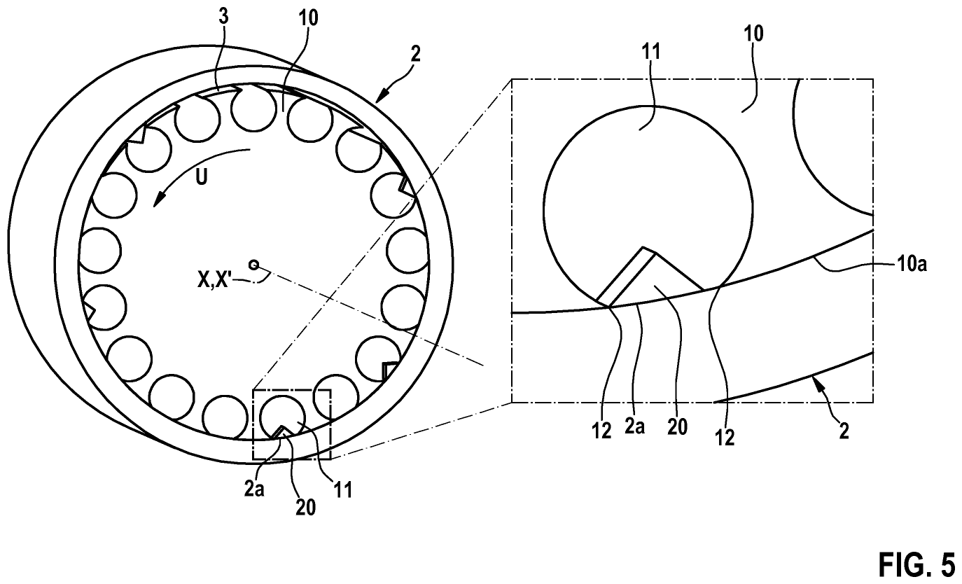

In accordance with a further embodiment (see for example FIG. 6) a tube made of the cobalt-chromium alloy L-605 according to ASTM F90 is processed by the method according to the invention. A tube 2 of this kind for example has an outer diameter of 1.800 mm and a wall thickness of for example 0.090 mm. The tube inner diameter is thus 1.620 mm. The inner surface 2a of the tube 2 again has undesirable plateau-like elevations 20, which consist of residues of baked-on tube drawing aids, cleaning solutions and materials of the inner tool (mandrel). These are for example compounds formed of carbon, iron and chlorine. In addition, there can be elements from the mandrel surface, such as zinc, and constituents of the lubricant, such as molybdenum, sulphur and phosphorus. Residues from previous tube cleaning processes, such as silicon carbide or aluminium oxide, are likewise possible. These partially baked-on contaminations 20 have a high adhesion to the inner surface 2a of the tube 2 and cannot be removed by conventional cleaning methods, such as pickling or sandblasting, without leaving behind any residue. In a tube 2 of this kind, 2 m long, a cylindrical tool 10 according to FIGS. 2 and 3 is inserted and is then arranged in the interior 3 of the tube 2, as shown in FIGS. 4 and 5.

The outer diameter of this structured cylinder or tool 10 made of hard metal is preferably 1.550 mm. The two tube ends of the tube 2 are preferably clamped in an apparatus in such a way that excessive tube bending is largely avoided by steady rests running concurrently. A coil 40 operated with alternating current is placed around the outer side of the tube 2 and is indicated schematically in FIG. 6 by a dashed circle. By integration of one or two for example rod-shaped permanent magnets in the interior of the cylindrical tool 10, there is a rotational movement of the cylindrical tool 10 about the rotation or cylinder axis x′ of the tool 10 when the coil 40 is started up. The rotational speed can be varied by the electrical parameters with which the coil 40 is operated. Both the cutting edges 12 and the surface structure 13 situated on the outer side 10a of the tool 10 provide a uniformly micro-structured tube interior 3 that is free from foreign material. This surface state, now provided, of the inner surface 2a of the tube 2 leads advantageously to a more uniform material removal of the process steps following the laser cutting, such as pickling and electropolishing. The entire tube inner surface or inner surface 2a of the tube 2 is treated by a longitudinal displacement of the coil 40 in the direction of the longitudinal axis x of the tube.

The embodiment according to FIG. 6 also offers the possibility of performing the material removal not over the entire tube length by way of a specific positioning of the coil 40. It is thus made possible to reduce the wall thickness from the inner surface 2a of the tube 2 in some sections. The internally structured tube 2 thus produced makes it possible to manufacture stent designs with wall thicknesses that are variable over the stent length.

The possibility of the inner processing according to the invention of tubes provides the following advantages: Firstly, an improvement of the semi-finished product quality leads to significant reductions of the rejection rate of the end products. Furthermore, the technical outlay for the post-processing processes (for example sandblasting of laser-cut stents) advantageously reduces. The standardisation of tube inner surfaces also enables a more uniform material removal of the possible subsequent chemical and electrochemical processes. This in turn allows an extension of the process limits. Extremely thin-walled stents can thus be produced for new fields of application (for example cranial).

The more uniform starting surface also leads to a greater uniformity of the final stent inner surfaces. This results in a higher fatigue strength associated with a greater medical device reliability.

With longer processing time, tube wall thicknesses which cannot be produced by conventional tube drawing techniques can additionally be provided.

Furthermore, in particular with use of the cylindrical tool 10, locally delimited changes to the tube inner diameter are possible. This opens up the possibility of producing stents of variable wall thickness.

1. A method for processing an inner surface of a seamlessly drawn tube, comprising:
inserting a tool into an interior the tube, wherein the tool has an outer side including multiple flow channels in the form of grooves arranged adjacently in a circumferential direction (U) and wherein each groove forms at least one cutting edge, and
moving the tool in the interior of the tube along a longitudinal axis (x) of the tube while simultaneously rotating the tool in the circumferential direction (U) of the tool by applying a gaseous medium (G) to the tool and/or by acting on the tool with an alternating magnetic field to remove contaminations of the tube which protrude from the inner surface.

2. The method according to claim 1, wherein the flow channels each have a curved profile.

3. The method according to claim 1, wherein the tool is spherical or ellipsoidal.

4. The method according to claim 1, wherein the tool is cylindrical.

5. The method according to claim 4, wherein the flow channels (11) each extend in a direction (D) which is skewed relative to a cylinder axis (x′) or rotation axis (x′) of the tool.

6. The method according to claim 1, wherein the flow channels have a circle segment shape in cross-section.

7. The method according to claim 1, wherein the outer side the tool has an abrasive surface structure.

8. The method according to wherein during the movement along the longitudinal axis (x), the tool is moved back and forth as a result of sides of the tool facing away from one another being acted on alternately by the gaseous medium (G), or as a result of the alternating magnetic field being moved back and forth accordingly along the longitudinal axis (x) with respect to the tube.

9. The method according to claim 1, wherein the tool comprises at least one permanent magnet for the movement and rotation of the tool by the alternating magnetic field.

10. The method according to claim 1, comprising rotating the tool at a particular position in a manner to vary a wall thickness of the longitudinal axis.

11. The method according to claim 1, wherein the tube is a blank for a medical implant device.

12. A device for processing an inner surface of a seamlessly drawn tube, comprising: a tool sized and configured to be inserted into the interior of the tube, wherein the tool has an outer side defining multiple flow channels in the form of grooves arranged adjacently to each other in a circumferential direction of the tool, wherein each groove on the outer side forms at least one cutting edge.

13. The device according to claim 12, comprising movement-generating means for moving the tool in the interior of the tube along a longitudinal axis (x) of the tube and for simultaneously rotating the tool in a circumferential direction (U) of the tool.

14. The device according to claim 12, comprising a movement-generating apparatus providing a gaseous medium (G) to move the tool in the interior of the tube along a longitudinal axis (x) to rotate the tool in a circumferential direction (U).

15. The device according to claim 12, wherein the flow channels each have a curved profile.

16. The method according to claim 1, wherein each groove forms at least two cutting edges.

17. The device of claim 12, comprising a movement-generating apparatus with a movable coil configured to generate an alternating magnetic field at the location of the tool to move the tool along a longitudinal axis (x) and/or to rotate it in a circumferential direction.

18. The device of claim 12, wherein each groove forms at least two cutting edges.

19. The device of claim 12, the tool having no mechanical connection to a movement-generating apparatus configured to move the tool longitudinally and rotationally in the tube.

20. The device of claim 12, wherein the tool is spherical and configured to exhibit tumbling movement in the tube.
